(19) United States
(12) Patent Application Publication
(10) Pub. No.: US 2025/0127159 A1
(43) Pub. Date: Apr. 24, 2025

(54) PAPER-BASED CRYOPRESERVATION OF MAMMALIAN CELL AGGREGATES

(71) Applicant: New York University in Abu Dhabi Corporation, Abu Dhabi (AE)

(72) Inventors: Mohammad A. QASAIMEH, Abu Dhabi (AE); Ayoub GLIA, Abu Dhabi (AE)

(21) Appl. No.: 18/917,636

(22) Filed: Oct. 16, 2024

Related U.S. Application Data
(60) Provisional application No. 63/591,570, filed on Oct. 19, 2023.

(51) Int. Cl. A01N 1/02; C12M 1/12; C12M 1/26; C12M 1/32; C12M 3/06; C12N 5/09

(52) U.S. Cl. CPC A01N 1/145; A01N 1/162; C12M 23/12; C12M 23/16; C12M 25/14; C12M 33/04; C12N 5/0693

(57) ABSTRACT

A system for a paper-based cryopreservation of mammalian cells includes a paper chip formed via a wax pattern printed on a paper substrate such that the wax pattern defines a plurality of hydrophilic wells that are separated from one another via a hydrophobic barrier. In addition, the system includes a microfluidics delivery device configured to load cells within the hydrophilic microwells of the paper chip. The microfluidics delivery includes a first component and a second component configured to receive the paper chip therebetween. At least one of the first and second components includes a first plurality of channels extending therethrough. Each of the plurality of channels extending therethrough along an axis from a first end aligned with a corresponding one of the microwells toward a second end, the second ends of the first plurality of channels converging at a first opening along a surface of the at last one of the first and second components so that cells are deliverable through the opening, through the first plurality of channels and to the microwells to load the cells therein.

### PRIORITY CLAIM

The present application claims priority to U.S. Provisional Patent Application Ser. No. 63/591,570 entitled “Paper-Based Cryopreservation of Mammalian Cell Aggregates” filed on Oct. 19, 2023, the entire disclosure of which is incorporated herein by reference.

### BACKGROUND

Cancer, characterized by uncontrolled cell division and genetic abnormalities, poses a significant challenge in medical research. Complex interactions between cancer cells and extracellular matrix (ECM) play a pivotal role in tumor progression and potentially lead to metastasis. Conventional preclinical methods for combatting this disease have relied on 2D cell culturing techniques, which fail to accurately replicate the spatial organization found in living tissue. This leads to altered cellular behaviors and molecular responses, thereby providing an inaccurate representation of in-vivo tumor conditions. For example, changes in substrate stiffness significantly impact gene expression profiles and drug responsiveness, while modulation of key signaling pathways affects vital cellular processes such as differentiation and migration.

Additionally, 2D cell cultures lack the crucial cell-cell and cell-ECM interactions that contribute to accurate cell signaling and morphology. The uniform nutrient and oxygen levels in 2D cultures also fail to account for factors such as tumor size and shape, which are critical determinants of the tumor microenvironment. As a result, the response observed in the lab based on these 2D model platforms leads to false predictions of clinical trial outcomes. The current pre-clinical testing processes in the pharmaceutical and biotech industries are highly resource-intensive, with a success rate to produce a drug at a mere 10%.

Three-dimensional (3D) in vitro tumor models offer a promising avenue for advancing the development of effective cancer drugs. These models have the potential to improve the predictability of drug sensitivity and toxicity in cancer. By recreating a more realistic 3D environment, these models can better capture the complexities of tumor biology and the interactions with its microenvironment. Spheroids, which belong to the scaffold-free systems sub-group, have gained attention in 3D cell culture. Tumor spheroids, which are self-assembled 3D aggregates of cancer cells, serve as robust models that replicate the complex intracellular and extracellular interactions, nutrient gradients, oxygen levels, and ECM composition observed in native tumors. Various techniques, such as the formation of neo-formed neural tubes from frog embryos, hanging-drop technology, and agarose molds, can be used to generate spheroids. However, incorporating spheroids into high-throughput screening platforms poses challenges, particularly in transferring them efficiently for detailed analysis.

Therefore, developing innovative spheroid fabrication platforms that facilitate their integration into high-throughput screening workflows while accurately mimicking the physiopathology of the tumor microenvironment is crucial. In particular, it is desired to develop innovative spheroid fabrication platforms that can be cryopreserved, stored for long durations of time, and can be used on demand within an off-the-shelf usage framework. These platforms will significantly enhance in-vitro drug screening and cell therapies.

### SUMMARY

The present disclosure relates to a system for a paper-based cryopreservation of mammalian cells. The system includes a paper chip formed via a wax pattern printed on a paper substrate such that the wax pattern defines a plurality of hydrophilic wells that are separated from one another via a hydrophobic barrier. In addition, the system includes a microfluidics delivery device configured to load cells within the hydrophilic microwells of the paper chip, the microfluidics delivery including a first component and a second component configured to receive the paper chip therebetween, at least one of the first and second components include a first plurality of channels extending therethrough, each of the plurality of channels extending therethrough along an axis from a first end aligned with a corresponding one of the microwells toward a second end, the second ends of the first plurality of channels converging at a first opening along a surface of the at last one of the first and second components so that cells are deliverable through the opening, through the first plurality of channels and to the microwells to load the cells therein.

In an embodiment, the microfluidics delivery device is scalable and includes two or more components, one of the two or more components configured as one of a male component, female component, and a neutral component.

In an embodiment, the first component is a female component printed to include a recess along a first surface thereof which, in an operative configuration faces toward the male component, the recess sized and shaped to receive the paper chip therein in an operative configuration, and the second component is a male component printed to include a protrusion extending from a first surface of the male component which, in the operative configuration faces toward the female component, the protrusion sized and shaped to correspond to the recess of the female component so that, in the operative configuration, the protrusion is received within the recess to secure and hold the paper chip therein.

In an embodiment, the paper chip is laminated to further reduce a likelihood of contamination.

In an embodiment, the paper chip is patterned via one of 3D printing resins inside of the paper substrate, mechanical pressing on the paper substrate, surface chemistry, and stenciling.

In an embodiment, the plurality of hydrophilic wells is plasma treated to improve liquid absorption and cell adhesion.

In an embodiment, the microwells are configured to support cryopreservation and 3D culture creating of cells.

In an embodiment, the plurality of hydrophilic microwells are formed by heating the paper chip so that the wax pattern printed on a first surface of the paper substrate is diffused toward a second surface of the paper substrate so that the diffused wax pattern acts as the hydrophobic barrier and defines each of the hydrophilic wells.

In an embodiment, the first opening is configured to engage a syringe for delivering the cells through the plurality of channels.

In an embodiment, the at least one of the first component and the second component including the first plurality of channels includes a second plurality of channels, each of the second plurality of channels extending therethrough along an axis from a first end aligned with a corresponding one of the microwells toward a second end, the second ends of the second plurality of channels converging at a second opening along a surface of the at last one of first component and the second component so that second cells are deliverable through the second opening, through the second plurality of channels and to the microwells to facilitate a co-culture of cells therein.

In an embodiment, the co-culture of cells may be achieved via one of stacking, folding, rolling, and origami architecture.

In an embodiment, a complex 3D co-culture with cryopreservation capabilities may be generated by combining the one of stacking, folding, rolling, and origami architecture with side-to-side co-cultures.

In an embodiment, the system further comprises a gasket that is sized, shaped and configured to encapsulate the paper chip therein, in a desired configuration.

In addition, the present disclosure relates to a method for paper-based cryopreservation of mammalian cells. The method includes developing a paper chip by printing a wax pattern on a paper substrate, the wax pattern defining a plurality of hydrophilic wells that are separate from one another via a hydrophobic barrier; and printing a microfluidics delivery device for loading cells to the microwells of the paper chip, the microfluidics delivery including a first component and a second component configured to receive the paper chip therebetween, at least one of the first and second components include a first plurality of channels extending therethrough, each of the plurality of channels extending therethrough, each of the first plurality of channels extending along an axis from a first end aligned with a corresponding one of the microwells toward a second end, the second ends of the first plurality of channels converging at a first opening along a surface of the at last one of the first and second components so that cells are deliverable through the opening, through the first plurality of channels and to the microwells to load the cells therein.

In an embodiment, the developing the paper chip includes laminating the paper chip to facilitate delivery of cells and reduce a likelihood of cross-contamination.

In an embodiment, the the plurality of hydrophilic wells is plasma treated to improve liquid absorption and cell adhesion.

In an embodiment, the microfluidics delivery device is scalable and includes two or more components, one of the two or more components configured as one of a male component, female component, and a neutral component.

In an embodiment, the microfluidics delivery device is one of contact, stamping and contact-less.

In an embodiment, the first component is a female component printed to include a recess along a first surface thereof which, in an operative configuration faces toward the male component, the recess sized and shaped to receive the paper chip therein in an operative configuration, and the second component is a male component printed to include a protrusion extending from a first surface of the male component which, in the operative configuration faces toward the female component, the protrusion sized and shaped to correspond to the recess of the female component so that, in the operative configuration, the protrusion is received within the recess to secure and hold the paper chip therein.

In an embodiment, the method further comprises loading the microwells of the paper chip with a first cell type via the first plurality of channels; culturing the cell-loaded paper chip to allow for spheroid formation; and cryopreserving the spheroid formation.

In an embodiment, the at least one of the first and second components is including first plurality of channels is 3D printed to include a second plurality of channels, each of the second plurality of channels extending along an axis from a first end aligned with a corresponding one of the microwells toward a second end, the second ends of the second plurality of channels converging at a second opening along a surface of the at last one of the first and second components.

In an embodiment, the method further comprises loading the microwells with a second cell type via the second plurality of channels to create a co-culture of cells therein.

In an embodiment, the method further comprises laminating the cell-loaded paper chip to seal the cell-loaded paper chip.

In an embodiment, the method further comprises encapsulating the cell-loaded paper chip via a gasket to ease handling and shipping thereof.

In an embodiment, the gasket is 3D printed to include a female component and a male component, each of the female component and the male component of the gasket being sized and shaped to correspond to a size and shape of the paper chip, in a desired configuration, such that the paper chip is receivable within the gasket in a sealing configuration.

In an embodiment, the gasket is configured to hold the cell-loaded paper chip therein in one of a planar, rolled, folded, and stacked configuration.

In an embodiment, the gasket includes a lock mechanism.

### BRIEF DESCRIPTION OF THE DRAWINGS

The patent or application file contains at least one drawing/photograph executed in color. Copies of this patent or patent application publication with color drawing(s)/photograph(s) will be provided by the U.S. Patent and Trademark Office upon request and payment of the necessary fee.

FIG. 1 shows a side view of a system for paper-based cryopreservation according to an exemplary embodiment of the present disclosure.

FIG. 2 shows a plan view of a wax-pattern printed on a filter paper, according to the exemplary system of FIG. 1.

FIG. 3a shows a cross-sectional side view of the paper and wax-pattern, according to the system of FIG. 1, printed on a first surface thereof.

FIG. 3b shows a cross-sectional side view of the paper as the wax pattern, according to the system of FIG. 1, is diffused from the first surface toward the second surface.

FIG. 4 shows a graphical representation of the wax penetration through the filter paper according to a paper type and a wax thickness, according to the system of FIG. 1.

FIG. 5 shows a graphical representation of a wettability based on a wax thickness and a plasma exposure time.

FIG. 6 shows a perspective view of a microfluidics delivery device according to the exemplary system of FIG. 1, in a disassembled configuration.

FIG. 7 shows the microfluidics delivery device, according to the exemplary system of FIG. 1, in an assembled configuration.

FIG. 8 shows a female component of the microfluidics delivery device according to FIGS. 6-7.

FIG. 9 shows a male component of the microfluidics delivery device according to FIGS. 6-7.

FIG. 10 shows a female component of a microfluidics delivery device according to another exemplary embodiment of the present disclosure.

FIG. 11 shows a male component of a microfluidics delivery device according to another exemplary embodiment of the present disclosure.

FIG. 12 shows a female component of a microfluidics delivery device according to an alternate embodiment of the present disclosure.

FIG. 13 shows a male component of a microfluidics delivery device according to an alternate embodiment of the present disclosure.

FIG. 14 shows results of a paper characterization with food dye based on varying wax thicknesses and/or microwell sizes.

FIG. 15 shows results of paper testing with breast cancer cells based on a wax thickness and/or microwell sizes.

FIG. 16 shows a schematic drawing of a method according to an exemplary embodiment of the present disclosure.

FIG. 17 shows a plan view of a gasket according to an exemplary embodiment of the present disclosure, the gasket including a female component and a male component.

FIG. 18 shows results of an analysis of characterization of microwell sizing with and without lamination thereof.

FIG. 19 shows results of an analysis of a viability of growth and metabolic activity of various cell lines.

FIG. 20 shows results of an assessment of cell viability and metabolic activity after cryopreservation, in which two groups of cells were grown for 3 weeks and 4 weeks, respectively, cryopreserved, re-cultured and assessed.

FIG. 21 shows results of an analysis of testing a variety of drug treatments on spheroids that were grown normally and those that were cryopreserved according to the exemplary method of the present disclosure.

### DETAILED DESCRIPTION

The present disclosure may be understood with reference to the following description and the appended drawings. The present disclosure related to a system and method for cryopreservation of cells and, in particular, relates to a system and method for a paper-based cryopreservation improving high-throughput screening workflows while accurately mimicking the physiopathology of in-vivo cells. Exemplary embodiments of the present disclosure describe a system and method for cryopreservation comprising a paper chip including microwells that are configured to be loaded with cells via a microfluidics delivery device. In an exemplary embodiment, the paper chip is treated with a wax pattern to form an array of hydrophilic microwells separated from one another via a hydrophobic wax barrier to prevent cross-contamination therebetween.

In an exemplary embodiment, the microfluidics delivery device includes a male component and a female component configured to receive the paper chip therebetween, one of the male and female components including a plurality of channels extending therethrough to facilitate precise, localized cell loading within each of the microwells via, for example, a syringe. Following this loading process the paper chip may be sealed in a gasket to facilitate storage and/or delivery so that the loaded paper chip may undergo a culture phase yielding spheroids, subsequently followed by a cryopreservation step.

It will be understood by those of skill in the art that although the exemplary embodiments are described with respect to cancer cells, the system and method of the present disclosure supports the cryopreservation and 3D culture of any of a variety of cells including, healthy cells, primary cells, blood cells, etc. It is also respectfully submitted that cryopreservation is on the rise and may be used for the preservation of any of a variety of biological materials in any of a variety of research industries including, for example, egg and sperm, placenta and umbilical cord, stem cells, tissue biopsies, pathogens, genome banks, and IQ banks. The paper chips of the exemplary system and method described herein are 500 times smaller than conventional cryotubes, improving the ease of handling thereof for storage and analysis.

Paper has emerged as a highly promising substrate in the field of biomedical research, attracting increasing attention for its diverse applications. By modifying its physical and chemical properties, paper can serve as an alternative to traditional cell culture substrates. Its unique ability to be assembled into three-dimensional (3D) structures makes it an excellent candidate for mimicking the complex microenvironment of living cells. Paper is a remarkable alternative to traditional 3D cell culture platforms such as hydrogels and porous scaffolds. Indeed, paper technology may also be used as a cryopreservation platform while integrated with hydrogels, porous scaffolds, electronics and photonic elements such as nanoparticles, nanorods graphene, MXene, and other 1D materials. Paper exhibits a wide range of surface topography and internal porous microstructure, providing a unique capability to manipulate cell behaviors.

Additionally, paper possesses an inherent capacity for fluid absorption through capillary action due to its porous structure and large void volume ratio. This property enables cell migration within the paper scaffolds. In some studies, paper has been combined with hydrogels to construct 3D in vitro culture platforms. The paper fibers act as a robust mechanical support, serving as a framework to maintain the configuration of the thin and mechanically fragile soft hydrogel. Consequently, cell-laden paper systems can be handled repeatedly without significant disruption to cell behaviors. Furthermore, the thickness of the paper can be tailored to be ≤200 μm, ensuring adequate oxygen delivery to all cells residing within the system. Moreover, by stacking multiple paper units with different cell types in a defined spatial distribution, it becomes possible to recapitulate the native 3D architecture found in-vivo. The unique features of paper-based systems, which facilitate 3D cell culture, open up a wide range of possibilities for various studies.

For instance, researchers can manipulate the physical or chemical properties of the paper to control nutrient and oxygen diffusion, thereby developing in vitro models of normal or diseased tissue. These 3D cell culture models can be effectively utilized to investigate cell-drug interactions in a high-throughput manner, thereby enhancing the understanding of disease mechanisms and drug metabolism. Moreover, the exceptional absorbing ability of paper makes it an excellent carrier for small-volume vitrification, as it can reduce the amount of vitrification solution surrounding the cells. In the realm of 3D cell culture, paper has exhibited remarkable potential, enabling the development of precise models for both normal and diseased tissues.

Exemplary embodiments of the present disclosure show and describe a system and method in which paper is wax patterned to optimize cell loading and isolation within each defined microwell, ensuring controlled cell growth and spheroid formation while preventing cross-contamination. Laminating these microwells-referred to as U-wells-improved their mechanical stability and handling during repeated freeze-thaw cycles and cryopreservation. The paper platform, however, may also be left without sealing, or sealed with lamination, or sealed with a gasket and/or any other sealing mechanism, from one side or from all sides.

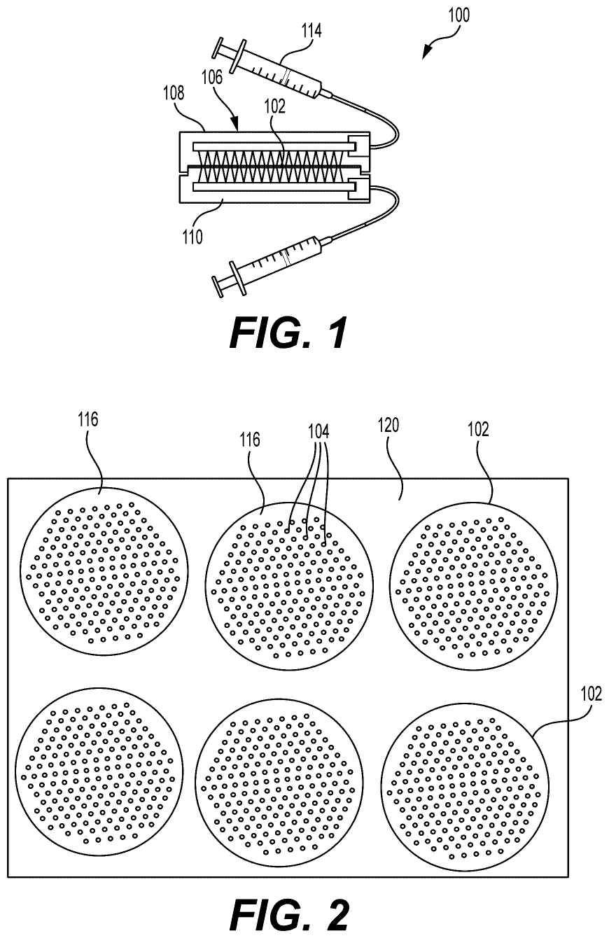

As shown in FIG. 1, an exemplary system 100 for a paper-based cryopreservation according to an exemplary embodiment of the present disclosure comprises a paper chip 102 that is configured to be loaded with cells via a microfluidics delivery device 106. According to an exemplary embodiment, the paper chip 102 is wax-patterned, as shown in FIG. 2, to define a plurality of hydrophilic microwells 104 (e.g., “U-wells”) configured to be loaded with cells. In an exemplary embodiment, the microfluidics delivery device 106 for loading of the cells to the paper chip 102 includes a female component 108 and a male component 110 between which the paper chip 102 may be sandwiched to facilitate precise cell loading. At least one of the female and male components 108, 110 may include a plurality of channels 112 through which cells may be loaded within the microwells 104 via a syringe 114.

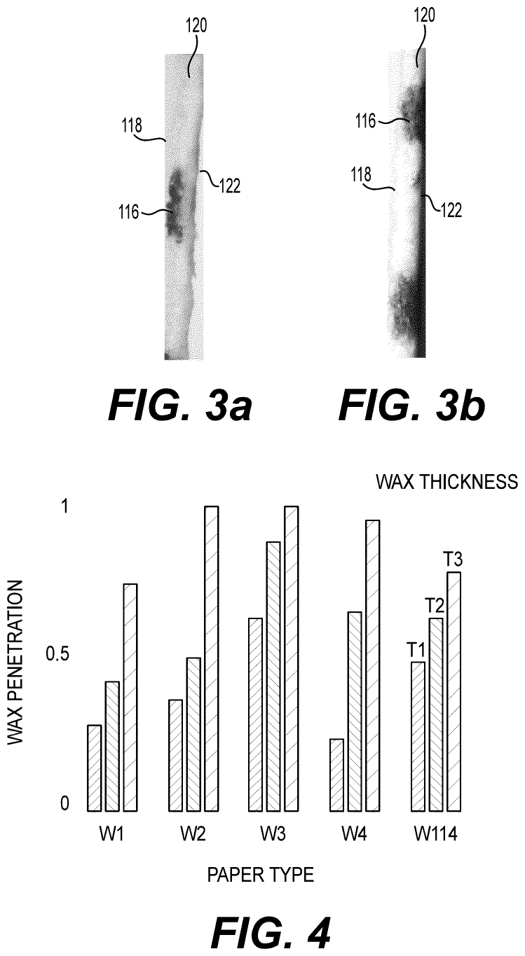

According to an exemplary embodiment, the paper chip 102 may be developed by printing a plurality of wax patterns 116 on a filter paper 120 to define an array of microwells 104, as shown in FIG. 2. In an exemplary embodiment, as shown in FIG. 3a, each wax pattern 116 may be printed on a first surface 118 of the paper 120. The paper 120 may then be heated to allow the wax to diffuse through the paper 120, from the first surface 118 toward a second surface 122, to define the hydrophilic wells 104, as shown in FIG. 3b. In an exemplary embodiment, the paper 120 may be heated at a temperature of approximately 120 degrees Celsius for 90 seconds. The wax forms a hydrophobic barrier 124 between each of the hydrophilic wells 104 to reduce the likelihood of contamination therebetween. In an exemplary embodiment, the microwells 104 may have a substantially U-shaped configuration. It will be understood by those of skill in the art, however, that the microwells 104 may have any of a variety of three-dimensional shapes. Furthermore, although the wax patterns 116 are described as forming “wells,” the paper chip 102 may include any of a variety of multidimensional features configured to be loaded with cells (cell aggregates) as described herein. For example, the paper chip 102 may include microchannels, microchambers, etc.

In an exemplary embodiment, each wax pattern 116 may be printed on the paper 120 in an array pattern so that each microwell 104 has a substantially circular configuration. It will be understood by those of skill in the art, however, that the microwells 104 may have any of a variety of configurations and may be separated from one another via a hydrophobic barrier having any of a variety of thicknesses. In another embodiment, for example, each wax pattern 116 may have a grid-like configuration. Each of the plurality of wax patterns 116 define a singular paper chip 102 so that upon heating of the paper 120 and formation of the microwells 104, each paper chip 102 may be cut from the paper 120. Each paper chip 102 may have any of a variety of shapes and sizes. In one example, each paper chip 102 may have a substantially circular shape. In another example, each paper chip 102 may have a substantially square and/or rectangular shape.

In an exemplary embodiment, to further reduce the likelihood of crosstalk or contamination between each of the microwells 104, each of the paper chips 102 may be laminated. In one example, each of the paper chips 102 may be laminated with, for example, a polyethylene terephthalate film using a laminator. The microwells 104 may also be plasma treated to enhance a wettability of each of the microwells 104, facilitating efficient cell seeding. In an exemplary embodiment, the plasma treatment may be applied for 30 to 60 seconds.

In an exemplary embodiment, in addition to wax, microwells may be created using 3D printed resins inside the paper, patterning using mechanical pressing, surface chemistry and stencil approaches.

An initial set of experiments were carried out to characterize the filter paper type use and optimize the wax-patterning process, circular patterns 116 were printed to define each of the paper chips 102 with varying thicknesses of wax lines to define a plurality of microwells 104 thereon. The thicknesses used were ¼ pt (referred to as T1), ½ pt (T2), ¾ pt (T3), and 1 pt (T4). The size of the resulting microwells was determined by the heated lines of wax, which diffused both horizontally and vertically upon heating. These experiments are aimed at finding the optimal diameter for cell storage in wells with minimal leakage, initially using dye and beads. The patterns were designed and printed onto different types of filter papers, specifically Whatman 1, 2, 3, 4, and 114. As shown and described above in regard to FIG. 3a, the printed wax created a layer only on the top side of the paper. The papers were then placed on heated plates to melt the wax through to the bottom side, forming a 3D barrier, as shown in FIG. 3b. The varying wax thicknesses resulted in varying levels of vertical diffusion (e.g., from the first surface 118 toward the second surface 122) and horizontal diffusion resulting in resulting in various microwell 104 sizes.

The temperature and duration were initially optimized at 120° C. for 90 seconds, with the filter papers placed on the heated plates accordingly. After removal, each wax pattern 116 was horizontally cut at the center, and a microscopic image of the wax was captured. This process was repeated for three samples and three different thicknesses of wax circles for each filter paper type. Using the captured microscopic images, the ratio between the horizontal distance the wax traveled on the bottom of the paper and the distance traveled on the top of the paper was measured.

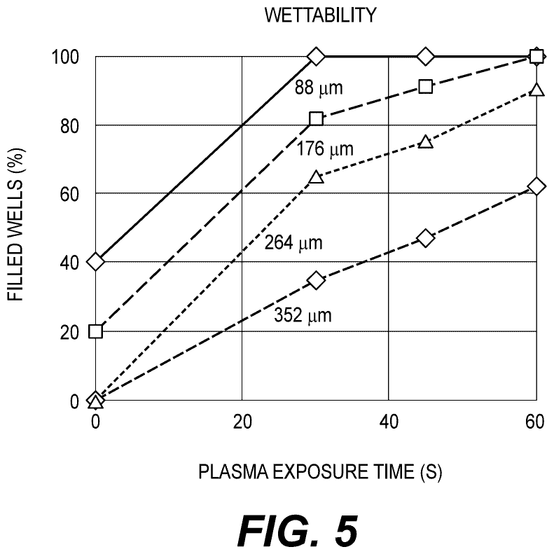

A ratio closer to 1 indicated more vertical wax diffusion through the paper, indicating a more preferable diffusion. Analyzing the results, as shown in FIG. 4, it was evident that the most suitable paper for wax melting using heated plates is Whatman Grade 2 filter paper (W2), as it exhibited ratios closest to 1 across the three different thicknesses. Next, as shown in FIG. 5, wax line thicknesses of ¼, ½, ¾, and 1 pt (corresponding to 88.19, 176.38, 264.57 and 352.76 μm, respectively) were used to characterize the correlation between the duration of plasma exposure (0, 30, 45, 60 s) and its resulting microwell permittivity. Results also suggested that ¾ pt wax line thickness produces a smaller and more isolated microwell (minimal cross talk and increased chance for cell aggregation), while preserving 90% microwell permittivity when exposed to plasma for a duration of 60 s.

It will be understood by those of skill in the art that although studies have revealed certain results in terms of filter paper used, a wax thickness, plasma exposure, etc., the system 100 of the present disclosure may utilize any of a variety of filter papers (e.g., fibrous substrates) having any of a variety of wax patterns 116 and wax barrier thicknesses that are heated and treated at any of a variety of temperatures and/or time periods to develop the paper chip 102 including hydrophilic microwells 104 separated from one another via the hydrophobic barrier (e.g., wax). It will also be understood by those of skill in the art that although the system 100 and, in particular, the paper chip 102, has been specifically shown and described as including a wax pattern 116, the paper chip 102 may be formed via 3D printing any of a variety of hydrophobic materials on/within the paper to define the hydrophilic microwells 104.

Upon formation of the paper chip 102, as described above, the microfluidics delivery device 106 (“Spherobox”) may be utilized for precise cell loading within the microwells 104. Spherobox enhances the accuracy and reproducibility required for high throughput drug screening. Traditional spheroid creation methods face challenges such as cross-contamination, inconsistent cell seeding, and difficulties in integrating these models into scalable workflows. These challenges often hindered the ability to accurately replicate the complex tumor microenvironment, a crucial factor for effective drug testing and personalized medicine. By offering precise, reliable, and scalable cell seeding into optimized U-well platforms, the Spherobox resolves these challenges.

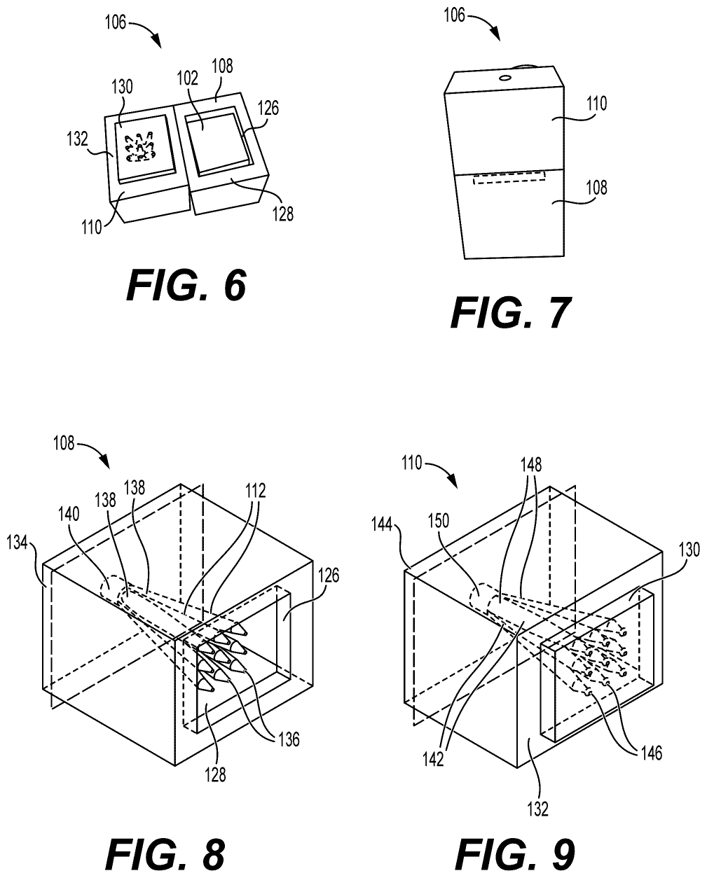

As described above, the microfluidics delivery device 106 is configured to facilitate loading of cells within the microwells 104 of the paper chip 102. According to an exemplary embodiment shown in FIGS. 6-9, the microfluidics delivery device 106 is comprised of the female component 108 and the male component 110. Although the exemplary embodiments show and describe the female and the male components 108, 110, it will be understood by those of skill in the art, however, that the microfluidics delivery device 106 is scalable, and may be formed of two or more parts in which one part may be configured as male, female or neutral. The microfluidics delivery device 106 may also be configured to be contact, stamping or contact-less such as, for example, injection deposition of cells in an inkjet-like approach. According to an exemplary embodiment, the components of the microfluidics delivery device 106 may be designed and created using CAD software and fabricated through stereolithography. It will be understood by those of skill in the art, however, that the microfluidics delivery device 106 may be 3D printed and/or manufactured using any of a variety of methods.

The female component 108 includes a recess 126 along a first surface 128 thereof which, in an operative configuration, faces toward the male component 110. The recess 126 is sized, shaped, and configured to securely receive the paper chip 102 therein, as shown in FIG. 6. The male component 110 includes a protrusion 130 extending along a first surface 132 thereof which, in the operative configuration faces toward the first surface 128 of the female component 108. The protrusion 130 is sized, shaped and configured to correspond to the recess 126 so that, in the operative configuration, the protrusion 130 is inserted into the recess 126 to securely hold the paper chip 102 therein, between the female and male components 108, 110, as show in FIG. 7. As will be described in further detail below, the female and the male components 108, 110 are configured to hold the paper chip 102 and allow for controlled cell seeding to improve spheroid formation consistency and significantly enhance the reproducibility of experimental outcomes, making it an indispensable tool in the development of more effective cancer therapies.

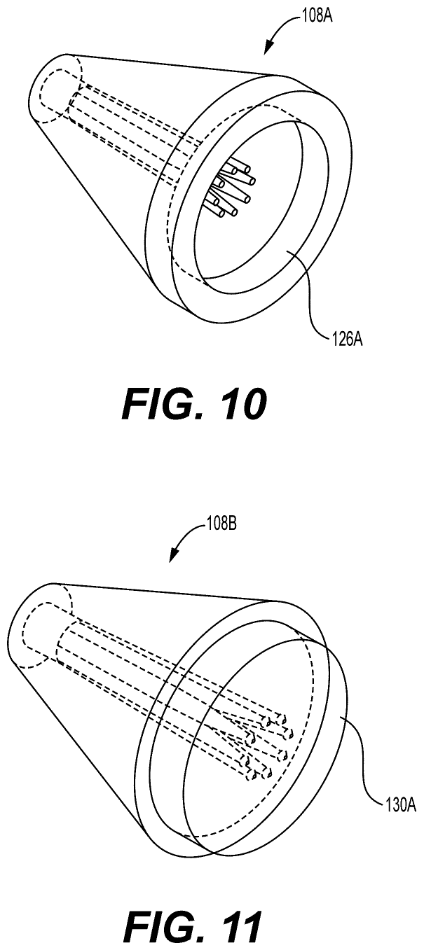

In an exemplary embodiment, the recess 126 and the protrusion 130 are sized and shaped to correspond to the size and shape of the paper chip 102. For example, in an embodiment in which the paper chip 102 is square, the female and male components 108, 110 and, in particular, the recess 126 and the protrusion 130, may be correspondingly square-shaped. In another example, as shown in FIGS. 10-11, where the paper chip 102 has a substantially circular shape, female and male components 108A, 110A and, in particular, a recess 126A and a protrusion 130A, respectively, may be correspondingly circular.

In an exemplary embodiment, one of the female component 108 and the male component 110 may include a first plurality of channels 112 extending therethrough so that fluid (e.g., cells) may be delivered through the channels 112 to the microwells 104. For example, as shown in FIG. 8, the female component 108 may include the plurality of channels 112, each of the channels 112 extending through the female component 108 from the first surface 128 thereof to a second surface 134 thereof, which opposes the first surface 128. Each of the channels 112 extends along an axis from a first opening 136 along the first surface 128, which is aligned with a corresponding one of the microwells 104 of the paper chip 102 received between the female and male components 108, 110, to a second opening 138.

The second openings 138 of the channels 112 converge at an opening 140 along the second surface 134, the opening 140 sized, shaped, and configured to engage or otherwise receive, for example, a portion of a syringe 114 via which cells may be pumped through the channels 112 to the microwells 104. Although the example describes the plurality of channels 112 as extending through the female component 108, it will be understood by those of skill in the art that the first plurality of channels 112 may similarly extend through the male component 110 to load the cells within the microwells 104 of the paper chip 102 received between the female and male components 108, 110 of the microfluidics delivery device 106.

In a further exemplary embodiment, however, the other one of the female and male components 108, 110 may include a second plurality of channels 142 extending therethrough. It will be understood by those of skill in the art that it may be desirable to have a set of channels 112, 142 extending through each of the female and the male components 108, 110 where it is desired to load a second type of cells to the microwells 104 to facilitate a co-culture of cells. For example, where the first plurality of channels 112 extends through the female component 108, the second plurality of channels 142 may extend through the male component 110 from the first surface 132 thereof to a second surface 144 thereof, which opposes the first surface 132. Similarly to the first plurality of channels 112, each of the second plurality of channels 142 may extend along an axis from a first opening 146 along the first surface 132, which is aligned with a corresponding one of the microwells 104 of the paper chip 102 received between the female and the male components 108, 110, to a second opening 148. The second opening 148 of the second plurality of channels 142 converge at a second opening 150 along the second surface 144. The second opening 150 may be sized, shaped, and configured to engage or otherwise receive, for example, a syringe 152 via which a second cell may be delivered loaded within the microwells 104.

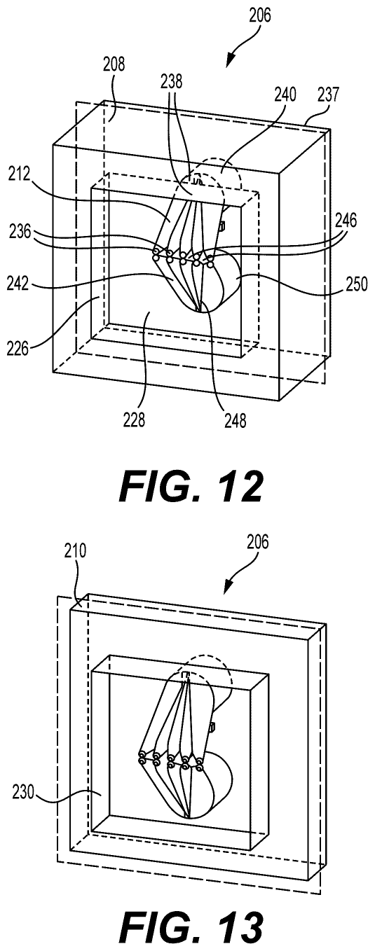

As shown in FIGS. 12-13, a microfluidics delivery device 206 according to another exemplary embodiment is similarly configured to facilitate co-culturing of cells during loading, the microfluidics delivery device 206 comprising a female component 208 and a male component 210. The female and male components 208, 210 are substantially similar to the female and male components 108, 110 of the microfluidics delivery device 106 described above, configured to hold a paper chip 102 securely therebetween via, for example, a recess 226 and a protrusion 230 substantially similar to the microfluidics delivery device 106. Rather than being configured to load a first and second cell onto the paper chip 102 via a same one of the female and male components, however, the first and second cells may be loaded to the paper chip 102 via first set of a plurality of channels 212 and a second set of a plurality of channels 242 extending through a same one of the female and male components 208, 210.

For example, the female component 208, 210 includes a first set of a plurality of channels 212, each channel 212 extending therethrough along an axis from a first opening 236 along a first surface 228 of the female component 208, which is aligned with a corresponding one of the microwells 104 of the paper chip 102, to a second opening 238. The second openings 238 of the channels 212 converge at an opening 240 along a second surface 234 of the female component 208. The first opening 240 is configured to engage and/or otherwise receive a portion of, for example, a first syringe carrying the first cells so that the first cells may be loaded to the microwells via the first set of channels 212. The second set of the plurality of channels 242 may also extend through the female component 208, each channel 242 extending along an axis from a first opening 246 along the first surface 228, which is aligned to a corresponding one of the microwells 104, to a second opening 248. The second openings 248 of the plurality of channels 242 converge at an opening 250 along the second surface 234 of the female component 208, the opening 250 configured to engage and/or otherwise receive, for example, a second syringe carrying a second cell, so that the second cells may be loaded to the microwells 104 via the second set of channels 242. The openings 240 and 250 are separated from one another along the second surface 234.

Although the first and second set of the plurality of channels 212, 242 are described as extending through the female component 208, it will be understood by those of skill in the art that the first and second plurality of channels 212, 242 may similarly extend through the male component 210 to facilitate a co-culture of cells, as shown in FIG. 13.

According to another exemplary embodiment, co-cultures may be performed by stacking, folding, rolling, and/or forming into favorable origami architectures. This is achieved with paper loaded with suspended cells, aggregates of cells, or a combination of both. In another exemplary embodiment, side to side co-cultures may also be combined with 3D stacking, folding, rolling approaches for generating complex 3D co-cultures with cryopreservation capabilities.

In a second set of experiments, the functionality of the Spherobox was evaluated through experiments testing the integrity of the wax barrier and the fluid diffusion within the paper platform using blue food dye. The ratio of the stained diameter to the wax microwell diameter was calculated to assess leakage, showing that heating the wax significantly reduced leakage and produced well sizes close to the optimal 1 mm. Additionally, 20 μm fluorescent beads, simulating cell size and shape, were delivered into wax-printed and plasma-treated U-wells with varying wax line thicknesses (¼, ½, and ¾ pt), confirming effective bead entrapment.

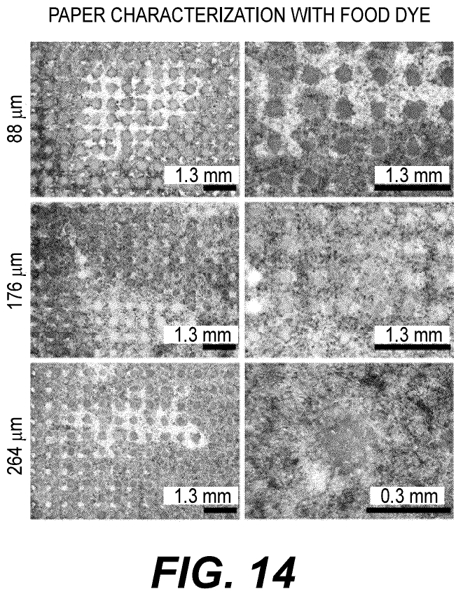

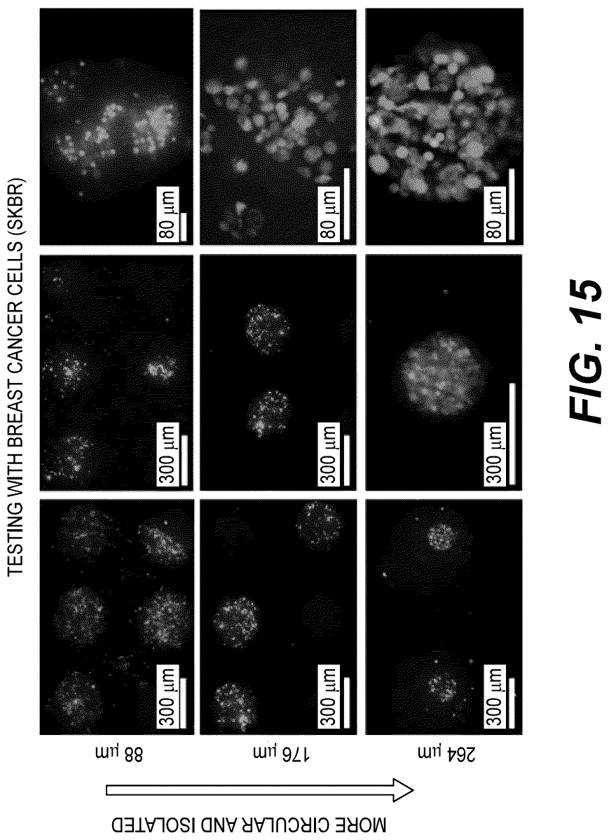

Further experiments involved delivering blue food dye and 20 μm fluorescent beads into wax-printed, plasma-treated paper matrices, as shown in FIG. 14. These tests confirmed the functionality of the wax barriers by evaluating fluid diffusion and bead entrapment across different wax line thicknesses. The results demonstrated that heating the wax minimized leakage, producing near-ideal well sizes. Moreover, as shown in FIG. 15, breast cancer cells (e.g., SKRB 3 cells) were passed through filter papers of varying pore sizes. Filter papers with an 11 μm pore size (Whatman 1, 2) retained significantly more beads compared to those with a 25 μm pore size (Whatman 114), supporting the selection of Whatman grade 2 filter paper for subsequent experiments due to its superior retention properties.

As will be described in further detail below, upon loading the paper chip 102 with cells using, for example, the microfluidics device 106, the cell-loaded paper chip 102 may be laminated or, in another embodiment, stored in a gasket 160. The gasket 160 may be specifically configured to encapsulate the paper chip 102 in any of a variety of configurations (e.g., planar, folded, rolled, stacked, etc.).

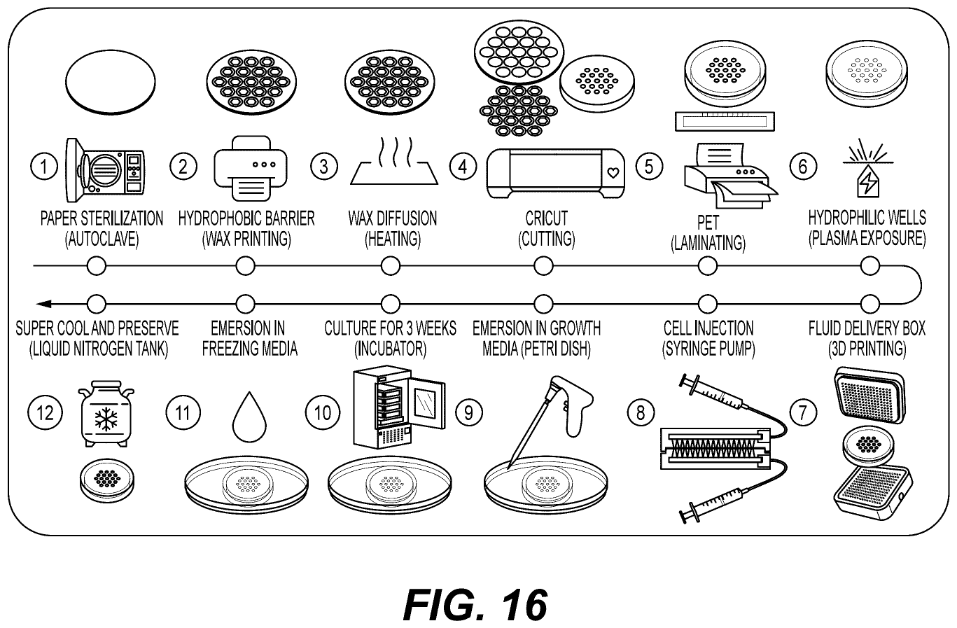

FIG. 16 shows an exemplary method 300 for paper-based cryopreservation according to the present disclosure. The method 300 comprises steps for paper chip 102 development, cell loading utilizing the microfluidics delivery device 106, and subsequent culturing and cryopreservation. In another exemplary method, the microwells creation and the cell deposition system can be incorporated in a roll-to-roll approach for scaling up the manufacturing capacity.

In a step 1, the paper 120 is sterilized via, for example, autoclaving. This sterilized paper 120 may then be wax-printed with the plurality of wax patterns 116, in a step 2. As described above, each of the wax patterns 116 may be printed on one side of the paper 120—e.g., the first surface 118-to define an array of microwells 104. In a step 3, the paper 120 may then be heated so that the wax patterns 116 diffuse through the paper 120, from the first surface 118 toward the second surface 122 so that the microwells 104 are formed therein. Each of the wax patterns 116 defines the hydrophilic microwells 104 by forming a hydrophobic barrier therebetween. As described above, each of the wax patterns 116 may be representative of a singular paper chip 102 so that each paper chip 102 may be cut from the paper 120, in a step 4. Each paper chip 102 may be laminated, in a step 5, to ease handling and enhance long-term stability. In a step 6, the microwells are plasma treated to improve liquid absorption and cell adhesion.

Upon development of the paper chip 102, as described above, the microfluidics delivery device 106 (or 206) may be 3D printed, in a step 7, to specifically include dimensions and vector parameters corresponding to the array of microwells 104, so that the paper chip 102 may be received and secured within the recess 126, between the female and male components 108, 110 thereof. One or more syringes 114 may be connected to one or more of female and the male components 108, 110 to load cells via, for example, the plurality of channels 112 and/or channels 142 within the microwells 104, in a step 8.

Once the cells have been loaded in the microwells 104 of the paper chip 102, the cell-loaded paper chip 102 may by immersed in growth media, in a step 9, and cultured (e.g., in an incubator) for approximately three weeks, in a step 10, to allow for spheroid formation. The spheroids are then preserved by immersion of the paper chip 102 in a freezing media, in a step 11, so that they may be cryopreserved and stored for future use (e.g., in a liquid nitrogen tank) for future use, in a step 12.

Although the culturing is shown and described as being performed via immersion in the growth media, it will be understood by those of skill in the art that the culturing may be performed externally (e.g., in a flask or petri dish) or internally via the microfluidics delivery device 106, wherein the cell medium, the necessary nutrients, and the metabolic products may be exchanged with full control by connecting the microfluidics delivery device to a syringe pump.

In an exemplary embodiment, a cell-loaded paper chip 102 may be sealed for ease of handling and/or shipping. In an exemplary embodiment the cell-loaded paper chip 102 may be sealed via a lamination. In another exemplary embodiment the cell-loaded paper chip 102 may be sealed via a gasket 160. The gasket 160 may be 3D printed or produced using any of a variety of other manufacturing methods, such that the gasket 160 is specifically sized and shaped to correspond to a desired configuration of the paper chip 102 to encapsulate the paper chip 102 therein in a tight-fit.

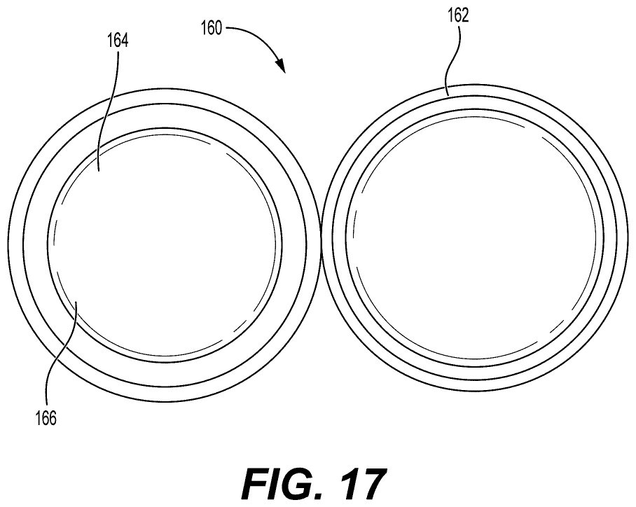

According to one exemplary embodiment, as shown in FIG. 17, the gasket 160 is formed of two parts including a female component 162 and a male component 164. The male component 164 may include a recess 166 formed therein, the recess 166 sized and shaped to receive the paper chip 102 therein. The female component 162 is configured to sealingly engage the male component 164 so that the paper chip 102 may be encapsulated therebetween. Although the gasket 160 is specifically described as being 3D printed, it will be understood by those of skill in the art that the gasket 160 may be printed and/or manufactured in any of variety of ways so long as the gasket 160 is configured to encapsulate the paper chip 102 as described.

Although the gasket 160 shown in FIG. 17 is shown as being configured to receive a planar sample of the paper chip 102, it will be understood by those of skill in the art that the gasket 160 may have any of a variety of shapes and configurations for encapsulating the paper chip 102 in any of a variety of configurations. In some embodiments, for example, a loaded paper chip 102 may be stacked, rolled, folded, etc. before or after cryopreservation and thawing procedures. The gasket 160 may be 3D printed, or otherwise manufactured, so that, in another embodiment, the gasket 160 is configured to hold/receive a folded, stacked, or a rolled sample. In another exemplary embodiment, the gasket 160 may be configured to fold and/or roll the sample therewithin. In yet another exemplary embodiment, the gasket 160 itself may have a folded configuration, holding the paper chip 102 substantially similarly to, for example, a business card case and/or a silicon wafer case.

In some embodiments, the gasket 160 may include a lock mechanism such as, for example, a snap mechanism, a sliding lock and/or a screw mechanism. The gasket 160 may be configured to be reusable and/or configured to dispense the paper chip 102, when desired. In another exemplary embodiment, the gasket 160 may be designed to be disposable. It will be understood by those of skill in the art that the gasket 160 may have any of a variety of configurations so long as the gasket 160 is configured to sealingly encapsulate the paper chip 102.

As described above, the microwell “U-well” platform of the present disclosure represents a significant advancement in 3D cell culture technology, specifically engineered to overcome the shortcomings of traditional 2D cultures and earlier 3D techniques in replicating the complex microenvironment of tumors for high-throughput drug screening and tumor model development. Traditional methods fall short in mimicking the intricate tumor ecosystem, crucial for effective cancer research and therapeutic interventions. Furthermore, these conventional techniques often grapple with issues like crosstalk and contamination between wells, especially during cryopreservation, thus compromising the reliability and reproducibility of results. The U-well system, with its isolated and stable microwells, enables precise and reproducible spheroid formation, alongside effective cryopreservation, and subsequent drug testing, offering a more faithful reproduction of the tumor microenvironment and enhancing the scalability of solutions for cancer research and personalized medicine.

### Characterization of Microwells

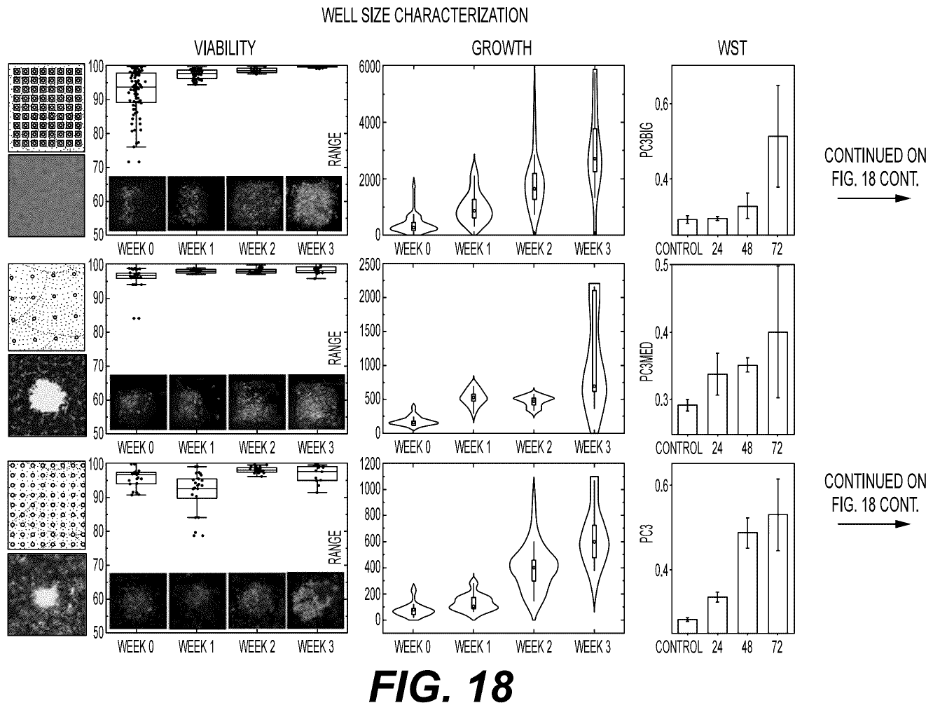

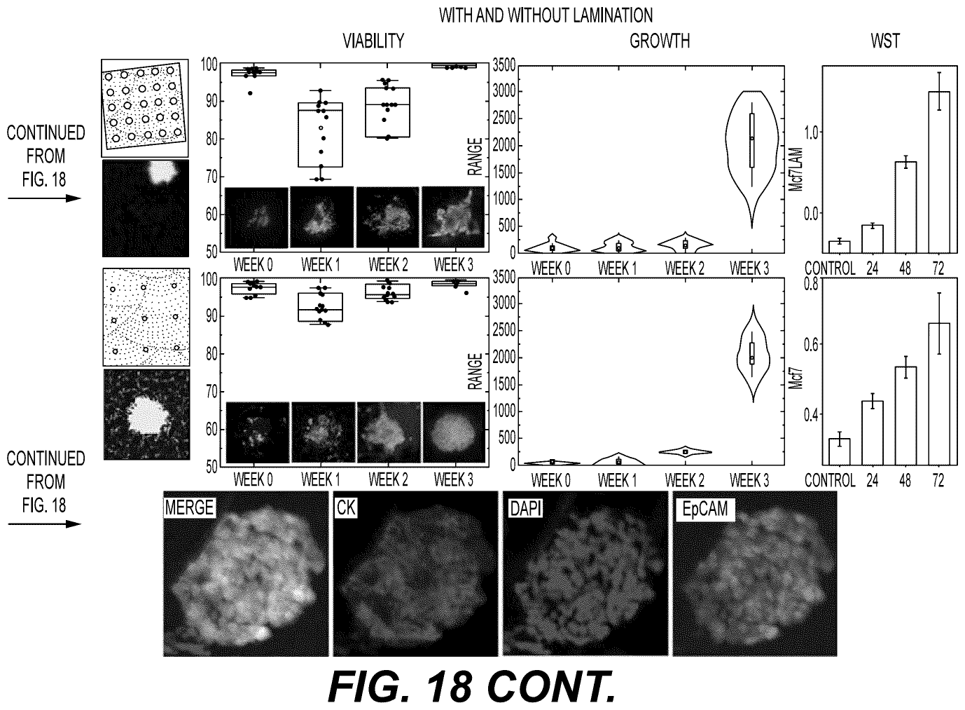

FIG. 18 provides a detailed analysis of how variations in well sizes—1 mm, 0.5 mm, and 0.2 mm—affect cell viability, growth, and metabolic activity across a three-week period, illuminating the superiority of certain dimensions over others. In the 1 mm wells, cell viability is consistently high, affirming that larger wells foster a stable environment conducive to sustained cell survival. This environment appears to support robust cellular functions and prevents the significant decline often observed in smaller wells. Cells in the 0.5 mm wells also demonstrate high viability, suggesting that this size too is suitable for maintaining healthy cell conditions. In contrast, the 0.2 mm wells show a decline in viability starting from the first week, likely due to the reduced cell density affecting cell proliferation and communication—key factors for viability in a constrained space.

The growth analysis further corroborates these findings, with cells in the 1 mm wells showing a marked and consistent increase in numbers, indicative of an optimal environment for cell proliferation. The broader distribution observed in the third week underscores a vigorous proliferation, in stark contrast to the more restrained growth in the 0.2 mm wells during the initial weeks. These patterns agree with viability data, where the challenges related to smaller well sizes demonstrate in initial growth delays but eventually resulting in improvement by the third week.

The metabolic activity across the wells, assessed via the WST-1 assay, aligns with growth and viability trends. The highest metabolic activity observed in the 1 mm wells parallels the enhanced cell count and sustained viability, underscoring the conducive environment these wells provide. Conversely, the 0.2 mm wells exhibit the lowest metabolic activity, corresponding to their diminished growth and viability. The introduction of lamination to the U-well design aims to refine the system further by enhancing packaging and reducing contamination risks, critical for applications involving primary cells. The comparative study between laminated and non-laminated U-wells seeded with MCF7 cells reveals that while both setups maintain high initial viability, non-laminated wells recover quicker from initial dips. This observation suggests that while lamination offers a controlled environment, reducing external variability and potential contamination, it might slightly impede early cell growth due to barriers it introduces.

### Platform Testing

Next, the scalability and effectiveness of the Spherobox and U-well platform for various cell lines used in pharmaceutical screening and vaccine production was evaluated. Seven key cell lines, including both cancerous and non-cancerous types, were seeded into U-wells and cultured for three weeks. Weekly assessments of viability, growth, and metabolic activity were performed to evaluate the performance of each line on the platform. Table 1 provides a summary of the cell lines tested, highlighting their cancer origin, metastatic potential, and importance in cell banking.

TABLE 1
Cell lines used in the study and their importance in cell banking

| | | | Importance | |
|---|---|---|---|---|
| Cell | | Metastatic | in Cell | |
| Line | Cell Origin | Potential | Banking | Ref |
| MCF7 | Breast Cancer | Low | High | |
| PC3 | Prostate Cancer | High | Moderate | |
| SKBR3 | Breast Cancer | Low to Moderate | High | |
| HCT | Colorectal | Moderate | Moderate | |
| | Cancer | | | |
| U87 | Glioblastoma | High | High | |
| | Cancer | | | |
| HeLa | Cervical Cancer | High | Very High | |
| MRC5 | Lung fibroblasts | Non-cancerous | High | |

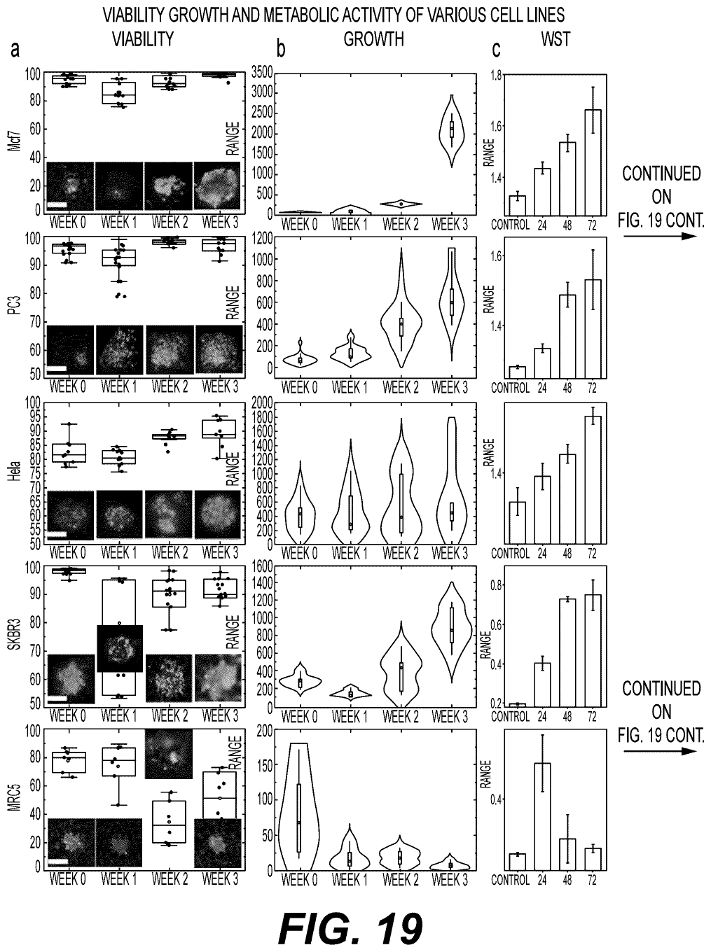

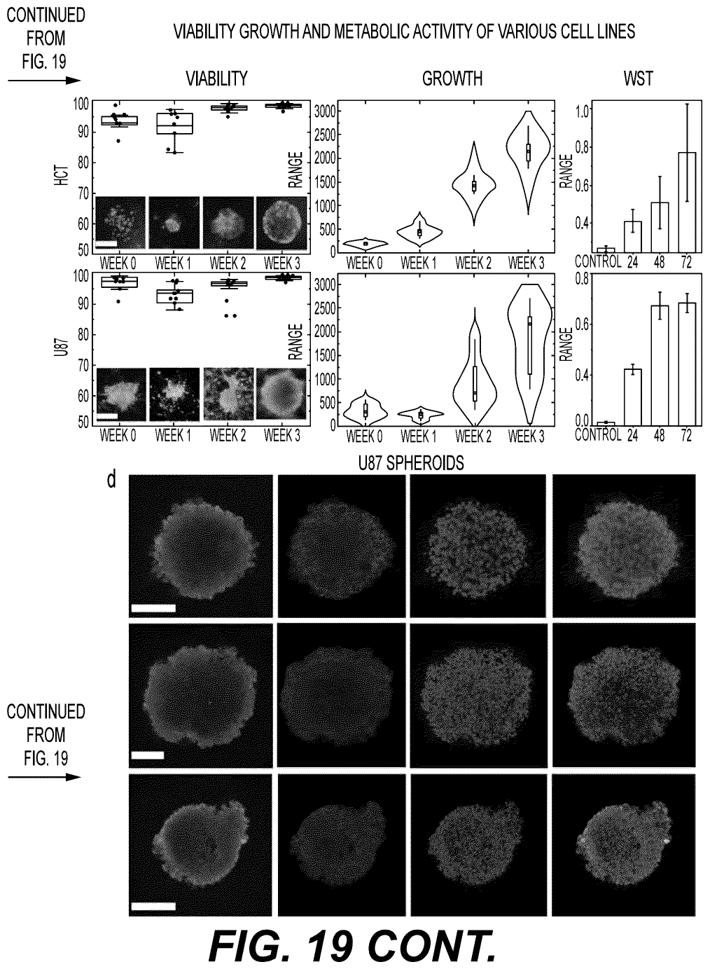

As shown in FIG. 19, initial viability across all cell lines was above 80% in week 0, though MCF7, PC3, SKBR3, and U87 exhibited temporary drops in week 1. Growth patterns identified three groups: cells with exponential growth (MCF7, PC3, SKBR3, HCT-15, and U87), cells with stationary growth (HeLa), and cells with exponential decline (MRC5). Metabolic activity assessments paralleled these findings, showing robust growth and adaptability for most lines, except for MRC5, which struggled with growth and viability on the platform.

Fluorescence imaging revealed diverse tendencies for 3D cluster formation. While MCF7, PC3, HeLa, and SKBR3 formed irregular 3D clusters, MRC5 failed to form such clusters. Conversely, HCT and U87 cells formed spheroids starting in week 2, reaching an average size of 300 μm by week 3. As shown in FIG. 19, most cell lines maintained viability above 80% during week 0. MCF7, PC3, SKBR3, and U87 experienced dips in viability during week 1 by 10%, 5%, 25%, and 5%, respectively, whereas HeLa and MRC5 showed no change, and HCT saw a slight increase. By week 2, viability increased for all lines except MRC5, which saw a sharp 45% decrease. By week 3, all cell lines except MRC5 showed viability above 90%, while MRC5 remained at 50%.

Growth patterns categorized cell lines into three groups. Exponential growth was observed in MCF7, PC3, SKBR3, HCT, and U87, reaching growth rates of 4000%, 800%, 300%, 1250%, and 733%, respectively. HeLa cells exhibited stationary growth despite high viability, and MRC5 showed exponential decline in growth, aligning with its reduced viability.

WST-1 metabolic activity results confirmed these trends. All lines except MRC5 demonstrated exponential increases in metabolic activity, indicating healthy and proliferating cells. MRC5 showed exponential decay, consistent with its poor growth and viability.

Fluorescent imaging indicated that most cell lines, including MCF7, PC3, HeLa, and SKBR3, formed irregular 3D clusters. MRC5 cells failed to form such structures. HCT and U87, however, began forming spheroids around week 2, achieving an average size of 300 μm by week 3. These results highlight the adaptability of certain cell lines to the U-well platform and suggest opportunities for optimizing culture and cryopreservation protocols tailored to specific cell types.

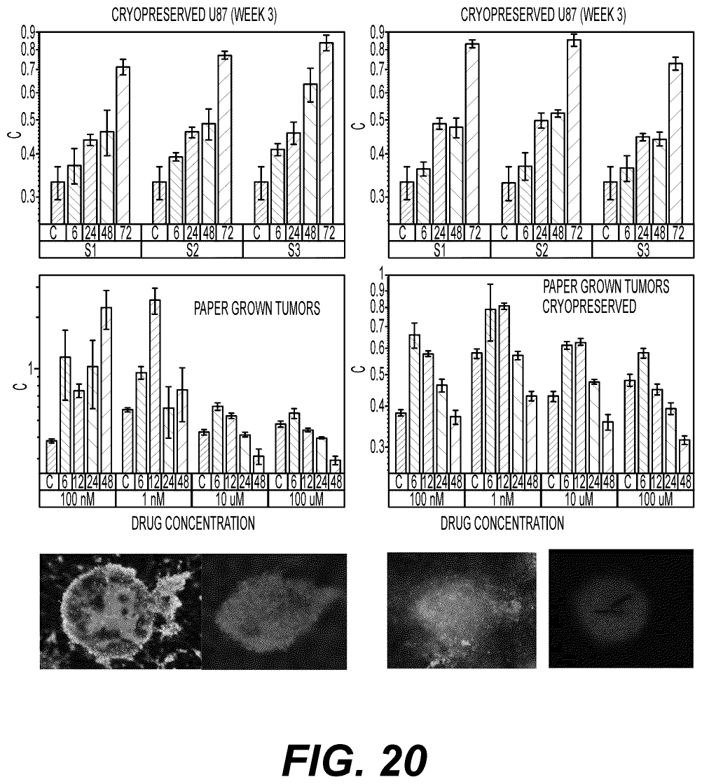

As shown in FIG. 20, cell cryopreservation was assessed via a study exploring the cryopreservation and drug testing capabilities of the platform using U87 spheroids. U87 spheroids were cultured for 3 and 4 weeks, then cryopreserved, thawed, and re-cultured for an additional week. The WST-1 assay gauged the metabolic activity of these spheroids, comparing their responsiveness based on initial growth duration before cryopreservation. Findings showed a gradual increase in metabolic activity post-thawing. However, 4-week spheroids displayed a temporary metabolic activity stagnation around the 48-hour mark, while 3-week spheroids showed continuous metabolic activity increase. This suggests initial growth duration before cryopreservation influences spheroid responsiveness, potentially impacting their behavior in subsequent drug testing. The 3-week spheroids, with uninterrupted metabolic activity increase, might exhibit heightened sensitivity or distinct response profiles compared to their 4-week counterparts. These findings highlight the importance of considering growth duration in cryopreservation and drug testing protocols, paving the way for further investigation into underlying mechanisms and implications for future applications.

U87 cells were divided into two groups, each seeded onto three U-well platforms (n=6) following Protocol-A. The first group was cultured for 3 weeks, and the second for 4 weeks. The U-well paper chip containing U87 spheroids was then carefully retrieved, rolled, and placed inside a standard cryotube. For successful cryopreservation, the spheroids were submerged in full growth medium supplemented with 10% dimethyl sulfoxide (DMSO, Sigma-Aldrich), then frozen at −1° C. per minute overnight using a Mr. Frosty container. The cryotube was then transferred to a −196° C. liquid nitrogen tank for long-term viability.

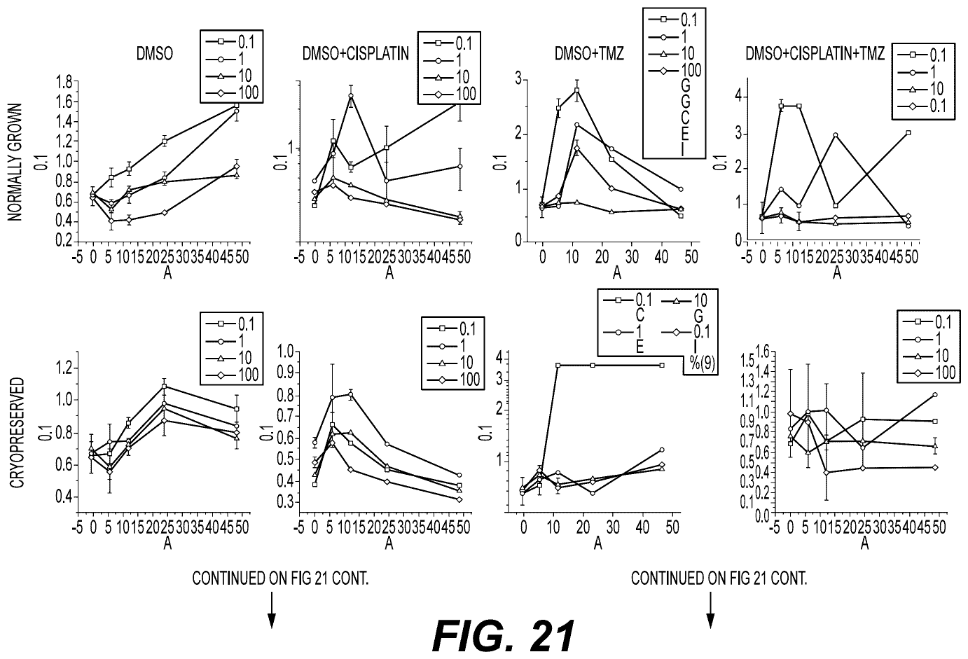

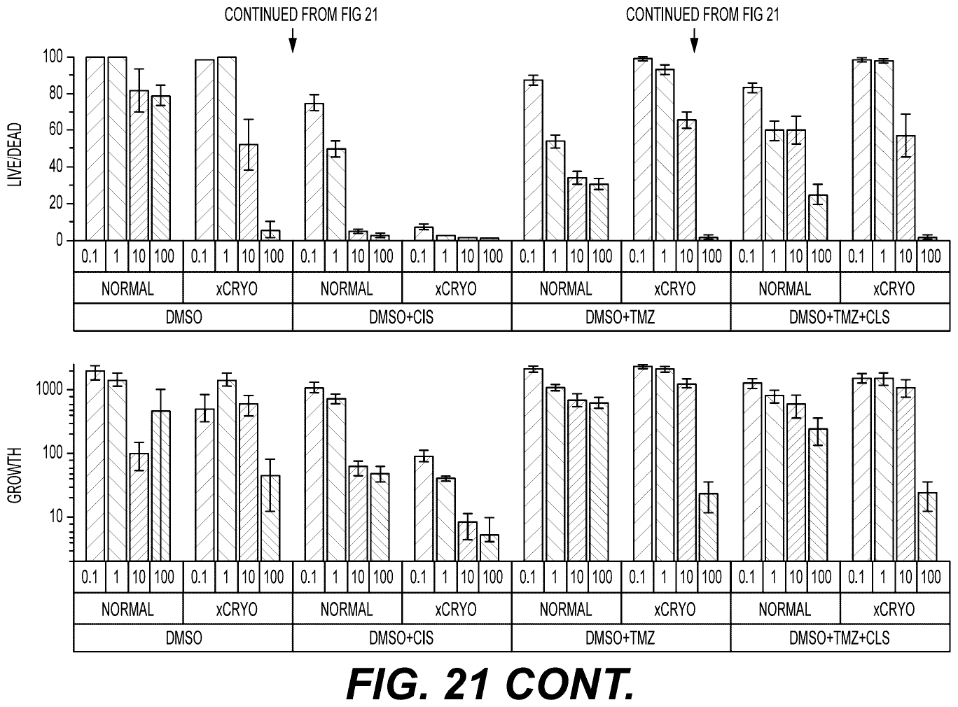

Upon thawing, the cryopreserved U87 spheroids were cultured for an additional week and subjected to the WST-1 assay at 6, 24, 48, and 72 hours. Results, as shown in FIG. 21, showed a gradual increase in optical density for both the 3-week and 4-week grown spheroids, indicating a considerable rise in metabolic activity. However, the metabolic activity of the 4-week grown spheroids temporarily stagnated around 48 hours before increasing again at 72 hours. In contrast, the 3-week grown spheroids displayed continuous metabolic activity increase without stagnation, suggesting differences in responsiveness based on growth duration. Further investigation is needed to understand these implications for future drug testing and research applications.

### Drug Testing

Glioblastoma multiforme (GBM) is a highly lethal central nervous system tumor known for its aggressive infiltration and drug resistance. Despite advances in treatment, the median survival of GBM patients remains low, with less than a 10% chance of surpassing a 5-year life expectancy. TMZ is the current first-line treatment for GBM, but its effectiveness is hindered by drug resistance mechanisms, including increased O₆-methylguanine-DNA methyltransferase (MGMT) activity. Given the complexity of GBM's molecular pathways, a combinatorial chemotherapy approach using multiple drugs, such as Cisplatin, has garnered interest, especially when used in combination with TMZ. A phase II trial demonstrated that combined Cisplatin and TMZ treatment significantly improved patient outcomes compared to TMZ alone, extending the time to progression and overall survival. FIG. 21 shows results of an analysis of the various drugs tested as treatment options.

In the context of this study, U87 spheroids—a commonly used model for GBM—were evaluated for their responsiveness to varying doses of Cisplatin, TMZ, and their combination, before and after cryopreservation, using the Spherobox. The spheroids were subjected to drug testing at varying concentrations, with DMSO serving as a solvent control. This experimental approach aimed to dissect the impact of both DMSO and drug compounds on the metabolic activity and viability of U87 spheroids.

Control tests were first conducted with DMSO at concentrations of 1%, 2.5%, 5%, 10%, and 20% (v/v) to assess its cytotoxic effects independently of drug action. The metabolic activity of the spheroids was monitored at 0, 6, 12, 24, and 48 hours, followed by a live/dead assay at 48 hours to assess cell viability. Results show that the normally grown spheroids indicated a linear increase in metabolic activity with 1% DMSO over 48 hours, while higher concentrations (5%, 10%, 20%) led to temporary reductions in metabolic activity at the 6-hour mark, followed by partial recovery. In contrast, cryopreserved spheroids exhibited increased sensitivity to higher DMSO concentrations, with metabolic activity declining sharply at 10% and 20% DMSO, reflecting reduced tolerance likely due to cryopreservation stress. Viability assays revealed excellent cell viability (>99%) for spheroids treated with 1% and 5% DMSO, while viability decreased for those exposed to 10% and 20% DMSO, particularly in cryopreserved samples, where viability dropped below 50% at 10% DMSO and to 5% at 20% DMSO.

Following the control studies, spheroids were exposed to increasing doses of Cisplatin (0.1, 1, 10, 100 μM), TMZ (100, 200, 400, 800 μM), and combined Cisplatin+TMZ treatments. Spheroid metabolic activity was measured at 6, 12, 24, and 48 hours using the WST-1 assay, and viability was assessed at 48 hours via the live/dead assay. Analysis of the normally grown spheroids revealed that Cisplatin significantly reduced metabolic activity at doses ≥10 μM, with viability dropping to less than 5% at 48 hours for 10 μM and 100 μM doses. Cryopreserved spheroids exhibited greater sensitivity to Cisplatin, with metabolic activity decreasing at doses as low as 0.1 μM. This suggests that cryopreserved spheroids are more responsive to lower Cisplatin concentrations, potentially due to alterations in cellular recovery post-thawing. In the case of TMZ, metabolic activity was suppressed at doses ≥100 μM for both normal and cryopreserved spheroids, with the most significant effects observed at 400 μM and 800 μM, where viability fell below 30%. Cryopreserved spheroids again demonstrated increased resilience, showing slightly higher metabolic activity and viability compared to normally grown spheroids at comparable doses of TMZ.

Combined Cisplatin+TMZ treatments demonstrated a dose-dependent reduction in metabolic activity, particularly at 4-fold and 8-fold combined concentrations. Cryopreserved spheroids exhibited slightly delayed but sustained responses, maintaining higher metabolic activity at 4-fold concentrations while showing significant suppression at 8-fold concentrations. Viability remained low (<5%) at high doses for both normally grown and cryopreserved spheroids, with cryopreserved spheroids showing enhanced resilience to the combination therapy. These results suggest that cryopreserved U87 spheroids are generally more responsive to lower doses of chemotherapeutic agents, such as Cisplatin and TMZ, but exhibit delayed responses at higher concentrations. This highlights the potential influence of cryopreservation on spheroid behavior, particularly in terms of drug metabolism and recovery. The observed trends in drug sensitivity across normally grown and cryopreserved spheroids underscore the importance of tailoring drug dosages and recovery periods in future 3D culture drug testing protocols.

This above-outlined study represents a significant advancement in the development of high-throughput drug screening platforms for cancer therapies, particularly through integration of 3D tumor models and cryopreservation within the present platform. The study shows that the microfluidics delivery device 106 (“Spherobox”) and the microwells 104 (“U-wells”) shown and described herein with respect to the exemplary system 100 and the exemplary method 300, provide a platform that is able to deliver a scalable, reproducible, and physiologically relevant solution for studying tumor biology and testing therapeutic agents.

A major innovation highlighted in this study is the successful integration of cryopreservation into the 3D tumor culture workflow. The ability to cryopreserve spheroids without significantly compromising their viability or responsiveness to drugs is crucial for scaling up experiments, enabling long-term storage, and improving reproducibility across different experimental setups. This feature is particularly relevant for personalized medicine, where patient-derived spheroids can be cryopreserved and tested with multiple therapeutic agents over extended periods. Results also demonstrate that the initial growth duration before cryopreservation plays a critical role in spheroid responsiveness, which emphasizes the need to optimize pre-cryopreservation protocols to maintain cellular integrity and functionality post-thaw.

The drug testing conducted with U87 spheroids provides important insights into the platform's utility for evaluating cancer therapies. The responsiveness of spheroids to Cisplatin and TMZ revealed several important findings. Notably, cryopreserved spheroids showed greater sensitivity to lower doses of Cisplatin compared to their normally grown counterparts, suggesting that cryopreservation may affect cellular pathways involved in drug uptake or metabolism. However, cryopreserved spheroids exhibited a delayed response at higher Cisplatin doses (10 μM and above), implying that the cryopreservation process may transiently impair certain metabolic or recovery pathways in the spheroids. This underscores the need for tailored protocols when using cryopreserved spheroids for high-throughput drug screening, as cryopreserved samples may exhibit distinct drug sensitivities.

For TMZ, both normally grown and cryopreserved spheroids showed effective suppression of metabolic activity at doses of 100 μM and higher. However, cryopreserved spheroids displayed slightly delayed responses to TMZ, with more substantial reductions in metabolic activity observed at higher doses (400 μM and 800 μM). These findings suggest that cryopreservation may induce subtle alterations in drug responsiveness, but overall, the efficacy of TMZ remains robust across both cryopreserved and non-cryopreserved samples. Additionally, the combination of Cisplatin and TMZ exhibited enhanced cytotoxicity, particularly at 4-fold and 8-fold doses, where significant reductions in viability were observed across both groups, suggesting that combinatorial chemotherapy approaches could be particularly effective in a 3D spheroid model.

Thus, the exemplary systems and method of the present disclosure provide transformative potential for cancer research and therapeutic testing. By integrating the advanced capabilities of 3D tumor modeling, cryopreservation, and high-throughput drug screening, it offers a robust and scalable solution for the development of more effective cancer therapies. The platform excels in replicating the complexities of tumor biology, including cell-cell and cell-matrix interactions, nutrient gradients, and drug resistance mechanisms, providing a more physiologically relevant model than traditional 2D cultures. Its ability to provide individualized insights into drug responsiveness makes SpheroMatrix an invaluable tool for both clinical research and pharmaceutical development, supporting the advancement of personalized medicine.

By leveraging this platform, researchers can better assess therapeutic efficacy, resistance, and potential combinatorial strategies for cancer treatment. The platform's successful application in evaluating Cisplatin and TMZ responses in U87 spheroids, for example, further underscores its utility, particularly in exploring combinatorial chemotherapy approaches. The ability to generate reproducible, sensitive drug response data across normally grown and cryopreserved spheroids highlights SpheroMatrix's versatility, paving the way for more precise and individualized therapeutic strategies. As the platform continues to be refined, its wide applicability and scalability ensure that SpheroMatrix will play a pivotal role in future cancer research, offering the potential to transform therapeutic testing and improve clinical outcomes.

It will be understood by those of skill in the art that the disclosure described and claimed herein is not to be limited in scope by the specific embodiments herein disclosed since these embodiments are intended as illustrations of several aspects of this disclosure. Any equivalent embodiments are intended to be within the scope of this disclosure. Indeed, various modifications of the disclosure in addition to those shown and described herein will become apparent to those skilled in the art from the foregoing description. Such modifications are also intended to fall within the scope of the appended claims.

What is claimed is:

1. A system for a paper-based cryopreservation of mammalian cells, comprising:
a paper chip formed via a wax pattern printed on a paper substrate such that the wax pattern defines a plurality of hydrophilic wells that are separated from one another via a hydrophobic barrier; and
a microfluidics delivery device configured to load cells within the hydrophilic microwells of the paper chip, the microfluidics delivery including a first component and a second component configured to receive the paper chip therebetween, at least one of the first and second components include a first plurality of channels extending therethrough, each of the plurality of channels extending therethrough along an axis from a first end aligned with a corresponding one of the microwells toward a second end, the second ends of the first plurality of channels converging at a first opening along a surface of the at last one of the first and second components so that cells are deliverable through the opening, through the first plurality of channels and to the microwells to load the cells therein.

2. The system of claim 1, wherein the microfluidics delivery device is scalable and includes two or more components, one of the two or more components configured as one of a male component, female component, and a neutral component.

3. The system of claim 1, wherein the first component is a female component printed to include a recess along a first surface thereof which, in an operative configuration faces toward the male component, the recess sized and shaped to receive the paper chip therein in an operative configuration, and the second component is a male component printed to include a protrusion extending from a first surface of the male component which, in the operative configuration faces toward the female component, the protrusion sized and shaped to correspond to the recess of the female component so that, in the operative configuration, the protrusion is received within the recess to secure and hold the paper chip therein.

4. The system of claim 1, wherein the paper chip is laminated to further reduce a likelihood of contamination.

5. The system of claim 1, wherein the paper chip is patterned via one of 3D printing resins inside of the paper substrate, mechanical pressing on the paper substrate, surface chemistry, and stenciling.

6. The system of claim 1, wherein the plurality of hydrophilic wells is plasma treated to improve liquid absorption and cell adhesion.

7. The system of claim 1, wherein the microwells are configured to support cryopreservation and 3D culture creating of cells.

8. The system of claim 1, wherein the plurality of hydrophilic microwells are formed by heating the paper chip so that the wax pattern printed on a first surface of the paper substrate is diffused toward a second surface of the paper substrate so that the diffused wax pattern acts as the hydrophobic barrier and defines each of the hydrophilic wells.

9. The system of claim 1, wherein the first opening is configured to engage a syringe for delivering the cells through the plurality of channels.

10. The system of claim 1, wherein the at least one of the first component and the second component including the first plurality of channels includes a second plurality of channels, each of the second plurality of channels extending therethrough along an axis from a first end aligned with a corresponding one of the microwells toward a second end, the second ends of the second plurality of channels converging at a second opening along a surface of the at last one of first component and the second component so that second cells are deliverable through the second opening, through the second plurality of channels and to the microwells to facilitate a co-culture of cells therein.

11. The system of claim 8, wherein the co-culture of cells may be achieved via one of stacking, folding, rolling, and origami architecture.

12. The system of claim 9, wherein a complex 3D co-culture with cryopreservation capabilities may be generated by combining the one of stacking, folding, rolling, and origami architecture with side-to-side co-cultures.

13. The system of claim 1, further comprising a gasket that is sized, shaped and configured to encapsulate the paper chip therein, in a desired configuration.

14. A method for paper-based cryopreservation of mammalian cells, comprising:
developing a paper chip by printing a wax pattern on a paper substrate, the wax pattern defining a plurality of hydrophilic wells that are separate from one another via a hydrophobic barrier; and
printing a microfluidics delivery device for loading cells to the microwells of the paper chip, the microfluidics delivery including a first component and a second component configured to receive the paper chip therebetween, at least one of the first and second components include a first plurality of channels extending therethrough, each of the plurality of channels extending therethrough, each of the first plurality of channels extending along an axis from a first end aligned with a corresponding one of the microwells toward a second end, the second ends of the first plurality of channels converging at a first opening along a surface of the at last one of the first and second components so that cells are deliverable through the opening, through the first plurality of channels and to the microwells to load the cells therein.

15. The method of claim 14, wherein developing the paper chip includes laminating the paper chip to facilitate delivery of cells and reduce a likelihood of cross-contamination.

16. The method of claim 14, wherein the plurality of hydrophilic wells is plasma treated to improve liquid absorption and cell adhesion.

17. The method of claim 14, wherein the microfluidics delivery device is scalable and includes two or more components, one of the two or more components configured as one of a male component, female component, and a neutral component.

18. The method of claim 14, wherein the microfluidics delivery device is one of contact, stamping and contact-less.

19. The method of claim 14, wherein the first component is a female component printed to include a recess along a first surface thereof which, in an operative configuration faces toward the male component, the recess sized and shaped to receive the paper chip therein in an operative configuration, and the second component is a male component printed to include a protrusion extending from a first surface of the male component which, in the operative configuration faces toward the female component, the protrusion sized and shaped to correspond to the recess of the female component so that, in the operative configuration, the protrusion is received within the recess to secure and hold the paper chip therein.

20. The method of claim 14, further comprising:
loading the microwells of the paper chip with a first cell type via the first plurality of channels;
culturing the cell-loaded paper chip to allow for spheroid formation; and
cryopreserving the spheroid formation.

21. The method of claim 14, wherein the at least one of the first and second components is including first plurality of channels is 3D printed to include a second plurality of channels, each of the second plurality of channels extending along an axis from a first end aligned with a corresponding one of the microwells toward a second end, the second ends of the second plurality of channels converging at a second opening along a surface of the at last one of the first and second components.

22. The method of claim 14, further comprising:
loading the microwells with a second cell type via the second plurality of channels to create a co-culture of cells therein.

23. The method of claim 14, further comprising:
laminating the cell-loaded paper chip to seal the cell-loaded paper chip.

24. The method of claim 14, further comprising:
encapsulating the cell-loaded paper chip via a gasket to ease handling and shipping thereof.

25. The method of claim 24, wherein the gasket is 3D printed to include a female component and a male component, each of the female component and the male component of the gasket being sized and shaped to correspond to a size and shape of the paper chip, in a desired configuration, such that the paper chip is receivable within the gasket in a sealing configuration.

26. The method of claim 24, wherein the gasket is configured to hold the cell-loaded paper chip therein in one of a planar, rolled, folded, and stacked configuration.

27. The method of claim 24 wherein the gasket includes a lock mechanism.
